(19) United States
(12) Patent Application Publication
(10) Pub. No.: US 2015/0335912 A1
(43) Pub. Date: Nov. 26, 2015

(54) SPLENIC IRRADIATION FOR THE TREATMENT OF NEUROLOGICAL DISORDERS

(71) Applicants: Loma Linda University, Loma Linda, CA (US); Loma Linda University Medical Center, Loma Linda, CA (US)

(72) Inventors: John H. Zhang, Colton, CA (US); Paul Krafft, Loma Linda, CA (US); Devin McBride, Alta Loma, CA (US); William Rolland, Sacramento, CA (US)

(21) Appl. No.: 14/716,781

(22) Filed: May 19, 2015

Related U.S. Application Data
(60) Provisional application No. 62/000,883, filed on May 20, 2014.

(51) Int. Cl. A61N 5/10

(52) U.S. Cl. CPC A61N 5/10

(57) ABSTRACT

Irradiation treatment can be used before, during, or after a surgical treatment or other injury to the brain to decrease or prevent subsequent injury. Injury to the brain during surgery can induce the infiltration of peripheral immune cells, including splenic immune cells, into the brain. The infiltration of immune cells can create acute elevations in brain edema and blood-brain barrier disruption thereby decreasing neurological recover and patient outcomes. Irradiation of peripheral immune cells, or the region of the body in which the cells are derived, can allow for inactivation of the cells and prevent infiltration into the surgical site, for example, the brain.

### CROSS-REFERENCE TO RELATED APPLICATION

Any and all priority claims identified in the Application Data Sheet, or any correction thereto, are hereby incorporated by reference under 37 CFR 1.57. The present application claims the benefit of U.S. Provisional Application No. 60/000,883, filed May 20, 2014. The aforementioned application is incorporated by reference herein in its entirety, and is hereby expressly made a part of this specification.

### FIELD OF THE INVENTION

Treatment of brain injuries with irradiation, including irradiation of the spleen to reduce injury and inflammation pre-surgery and/or post-injury.

### DESCRIPTION OF THE RELATED ART

According to a National Neurosurgical Procedural Statistics 2006 Survey, conducted by the American Association of Neurological Surgeons, 592,443 cranial surgeries were performed in that year alone. Cranial procedures always carry a degree of inevitable injury to the function of a normal brain. This may result in a prolonged or incomplete recovery in patients undergoing cranial surgery. Even the majority of healthy patients, patients referred to as physical status I and II by the American Society of Anesthesiologists, receive intensive or immediate care after elective craniotomy. This translates into longer hospital stays with significant implications for the patient. Furthermore, the considerable amount of health care expenditures related to surgical procedures is an important socioeconomic burden in the US.

To date, developing less invasive surgical methods and administering nonspecific postoperative care have been the predominant strategies for limiting surgically-induced brain injury (SBI), with relatively little research into understanding the underlying pathophysiology and targeting specific pathways. The complications of surgically-induced brain injury not only require vigilant postoperative care, but may also hinder the surgical approach to critical brain regions. Diminishing the perioperative risks of surgically-induced brain injury could expand the possibility for more complex and long-lasting neurosurgical interventions.

In the over half a million cranial surgeries that are performed annually in the US, a certain degree of inevitable injury to the functional healthy brain will occur. This inevitable injury may result in the patient's prolonged or incomplete recovery. Accordingly, preventing the development of surgically-induced brain injury may translate into enhanced post-operative recovery, thus reducing the individual, as well as the economic, burden related to surgically-induced brain injury.

Recent studies revealed a close linkage between acute brain damage and the subsequent inflammatory response by spleen-derived immune cells, e.g., splenocytes. Splenocytes have been shown to infiltrate the brain after experimental ischemic and traumatic brain injury. However, subjects who have undergone a surgical splenectomy have shown improved neurofunctional and morphological outcomes. Stroke and traumatic brain injury share various pathophysiological mechanisms with surgically-induced brain injury, and therefore the same treatment can possibly reduce injury for various brain disorders, as well as other systemic diseases. Currently, there are no specific efforts to prevent or attenuate surgical brain injury. Surgical brain injury is considered unavoidable and is being ignored by physicians and health care providers. A technique that can be used before an elective surgery or after an injury has occurred to provide a therapeutic benefit via reduced inflammation and immune cell infiltration of the injury can improve quality of life and outcome of patients.

### SUMMARY

There is a need for a technology that can be adopted by physicians and other surgeons before an elective surgery or after an injury has occurred to provide a therapeutic benefit through reduced inflammation and reduced immune cell infiltration of an injury. Additionally, the ability to further reduce the impact of surgery, improve the quality of life, and reduce the impact of post-operative care on the healthcare system is desirable. Therapies that cause inactivation of the spleen and their associated immune cells can be used for the treatment of brain injuries.

In accordance with one aspect, a method is provided of administering treatment prior to a surgical procedure comprising: providing a radiation source to apply gamma radiation to a target area discrete from a surgical site; irradiating the target area under the gamma radiation from the radiation source; inactivating an immune response generated from the target area; inflicting a primary injury at the surgical site; and reducing a secondary injury through inactivating the immune response from the target area. In some embodiments, the radiation source is a Cobalt-60 irradiation unit. In some embodiments, the gamma radiation is Cobalt-60. In some embodiments, the surgical site is a brain. In some embodiments, the method wherein the primary injury at the surgical site comprises acute brain injury. In some embodiments, the acute brain injury initiates the release of cytokines to stimulate sympathetic adrenergic output.

In some embodiments, the target area is a spleen. In some embodiments, the inactivated immune response is an inflammatory response by spleen-derived immune cells. In some embodiments, the secondary injury comprises an edema at the surgical site. In some embodiments, the target site comprises a plurality of sites in the patient's body. In some embodiments, reducing the secondary injury further comprises reducing are least one of edema, neurological defects, blood-brain barrier disruption, neuroinflammation, and neuronal cell death. In some embodiments, the method further comprises administering a one-time, low-dose gamma irradiation of the target area to reduce the secondary injury caused by the immune response.

In accordance with another aspect, a method is provided of treating surgically-induced brain injury before a brain surgery comprising: providing a radiation source to apply gamma radiation to a target area discrete from a brain surgery site; irradiating the target area under the gamma radiation from the radiation source; inactivating an immune response generated from the target area; performing a surgical procedure at the brain surgery site; inflicting a primary injury at the brain surgery site through performing the surgical procedure; and reducing a secondary injury through inactivating the immune response from the target area. In some embodiments, the radiation source is a Cobalt-60 irradiation unit. In some embodiments, the gamma radiation is Cobalt-60. In some embodiments, the primary injury at the brain surgery site comprises acute brain injury.

In some embodiments, the acute brain injury initiates the release of cytokines to stimulate sympathetic adrenergic output. In some embodiments, the target area is a spleen. In some embodiments, the inactivated immune response is an inflammatory response by spleen-derived immune cells. In some embodiments, the secondary injury comprises an edema at the brain surgery site. In some embodiments, the target site comprises a plurality of sites in the patient's body. In some embodiments, reducing the secondary injury further comprises reducing are least one of edema, neurological defects, blood-brain barrier disruption, neuroinflammation, and neuronal cell death. In some embodiments, the method further comprises administering a one-time, low-dose gamma irradiation of the target area to reduce the secondary injury caused by the immune response. In accordance with another aspect, a method is provided of treating surgically-induced brain injury as described herein.

### BRIEF DESCRIPTION OF THE DRAWINGS

FIGS. 1A-B illustrate the pathway of the brain spleen inflammatory coupling and the interference of splenic irradiation.

FIGS. 2A-C show the occurrence of brain edema and neurological deficits following surgically-induced brain injury.

FIGS. 3A-C show CD11b expression and localization following surgically-induced brain injury.

FIG. 4 shows CD3 expression following surgically-induced brain injury.

FIGS. 5A-C display gamma irradiation-induced changes in the spleen.

FIG. 6 shows the brain water content in sham surgery, surgically-induced brain injury, and surgically-induced brain injury with splenectomy.

### DETAILED DESCRIPTION

Surgically-induced brain injury or surgical brain injury typically includes unavoidable collateral damage to healthy brain tissue, caused by intraoperative dissection, retraction, and hemorrhage, which ultimately may manifest as neurological decline. In fact, the rate of procedure-associated major complications following craniotomy, such as the appearance of a new sensorimotor deficit or seizure disorder, was reported at between 10.3 and 13%. Endoscopic-assisted and stereotaxic procedures were developed to alleviate the extent of iatrogenic injury to adjacent brain structures, however with limited success. Even stereotactic radiosurgery (SRS), one of the least invasive procedures used by neurosurgeons, has the potential to evoke sensorimotor deficits and edema-induced ataxia in patients.

Surgical brain injury may occur despite administration of osmotic agents, diuretics, and steroids. Therefore, innovative therapies, which directly target the underlying pathophysiologic mechanisms of surgical brain injury, are needed to improve the patient's postoperative recovery.

One of the main complications of surgery-induced brain injury is the formation of brain edema. Surgery-induced brain injury develops by both, primary and secondary mechanisms of injury. Primary injury, inflicted directly by mechanical manipulation during surgery, is inevitable and irreversible. However, the delayed secondary development of localized brain edema has been shown to be a major postoperative complication of cranial procedures and potentially can be alleviated or avoided. Preclinical surgery-induced brain injury studies indicate that the brain water content of the resection border increases by as much as 3-4% during the first three postoperative days. A 4% increase in brain water content can translates into a 25% increase in tissue volume, leading to high intracranial pressure, hypoperfusion of neurons, and cell death. Therefore, it can be advantageous to reduce or eliminate the delayed secondary development of localized brain edema thereby reducing surgery-induced brain injury.

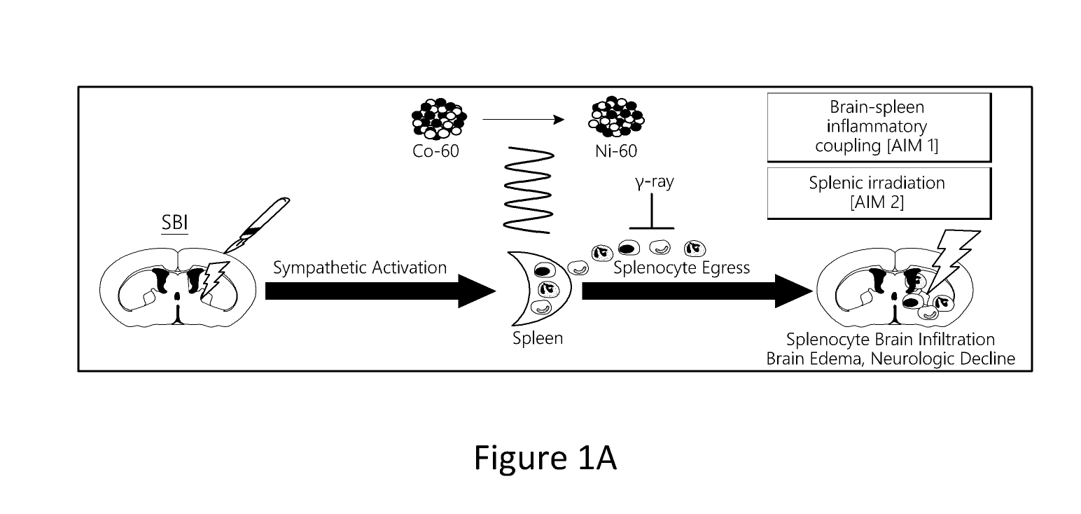

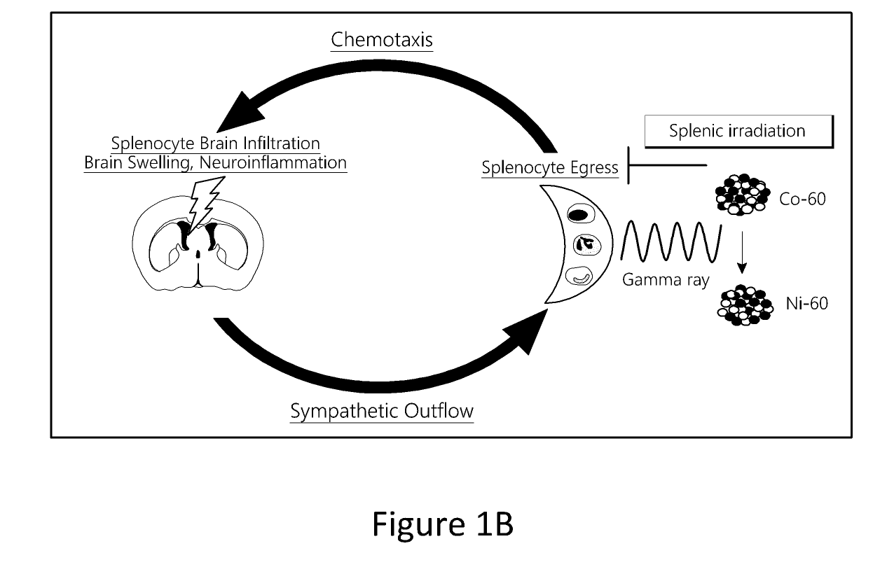

Recent studies demonstrated a close linkage between acute brain injury and the subsequent inflammatory response by spleen-derived immune cells. The spleen, a lymphoid organ, contains immune cells, splenocytes including monocytes and lymphocytes, as well as a population of granulocytes such as neutrophils and neutrophil-like cells. Brain-spleen inflammatory coupling describes the phenomenon of altered splenic function as a response to brain injury. Acute brain injury initiates the release of locally acting cytokines by damaged neurons and glial cells, which stimulate hypothalamic nuclei to increase sympathetic output as shown in FIGS. 1A-1B. This sympathetic adrenergic stimulation or activation facilitates egress of spleen-derived immune cells (e.g., splenocytes), into the circulation as well as the secretion of pro-inflammatory mediators by splenic macrophages. Acute brain injury increases sympathetic output and adrenergic stimulation of the spleen facilitates the release of stored lymphocytes, granulocytes, and monocytes.

Following acute brain injury, the spleen is activated through adrenergic pathways, which induces splenocyte egress into the injury area. Directed by chemotactic signals, these immune cells enter the brain through a compromised cerebral microcirculation, thereby aggravating blood-brain barrier leakage and edema formation. Brain-infiltrated lymphocytes can have a direct cell contact-dependent neurotoxic potential. Furthermore, activated immune cells can produce pro-inflammatory cytokines such as interleukins and tumor necrosis factor-α (TNF-α). TNF-α initiates extramitochondrial apoptotic pathways through caspase-8 activation. The activation of the apoptotic pathway results in brain injury through the cell changes and/or cell death that occurs.

Spleen-derived immune cells have a role in the injury progression following surgically-induced brain injury or other neurological disease or injury. FIGS. 1A-B illustrates the pathway of the brain-spleen inflammatory coupling and the interference of splenic irradiation. As illustrated in FIGS. 1A-B, surgically-induced brain injury can stimulate the release of splenocytes from the spleen. The splenocytes can infiltrate the brain, thereby causing blood-brain barrier disruption, vasogenic edema formation, and neuroinflammation, ultimately worsening the postoperative sequelae or condition. The splenocytes infiltration of the brain can be responsible for brain edema and neurological decline in patients after brain injury. In some embodiments, a close temporal relationship between cerebral edema formation and the large number of brain-infiltrated myeloid and lymphoid cells following experimental surgically-induced brain injury has been demonstrated. The delayed development of localized brain edema has been shown to be a major postoperative complication of cranial procedures.

Splenocytes can be involved in the progression of hypoxic-ischemic, traumatic brain injury, and surgically-induced brain injury. Inhibition of the peripheral immune response, through inhibition or reduction of splenocytes into the injured tissue area, has been found to reduce not only cerebral edema and inflammation but also can provided neuronal protection and improved functional outcomes for patients with hypoxic-ischemic and traumatic brain injury. In addition to infiltration of splenocytes into the brain during ischemic and traumatic brain injury, splenocytes have been shown to infiltrate the brain after surgically-induced brain injury. Thus, in some embodiments, temporarily inhibiting their viability can present a prospective therapeutic strategy for patients with hypoxic-ischemic, traumatic brain injury, and/or patients undergoing brain surgery.

Additionally, because of the elective nature of many neurosurgical procedures, surgically-induced brain injury is a prime candidate for preemptive therapies. Neuroprotection developed by pretreatment techniques, may avert the development of potentially irreversible pathophysiologic mechanisms, thereby guarding the susceptible brain from surgically-induced brain injury.

In some embodiments, surgical splenectomy can provide improved neurofunctional and morphological outcomes in subjects by preventing the infiltration of splenocytes into the brain area. Various brain disorders share various pathophysiological mechanisms with stroke, traumatic brain injury, and surgically-induced brain injury, and thereby the reduction or prevention of infiltration of splenocytes into the brain can possibly reduce injury for various brain disorders, as well as other systemic diseases. The implication of peripheral immune cells is also well documented in various central nervous system diseases, including acute brain injury, stroke, epilepsy, multiple sclerosis, movement disorders, and Alzheimer's dementia. Furthermore, irradiation of the spleen may be relevant not only for surgical brain injury but also for a multitude of other inflammatory central nervous system diseases and/or acute brain injury

Therapies to prevent or reduce the infiltration of splenocytes into the brain can be used before an elective surgery or after an injury has occurred. Accordingly, pharmacological blockade of adrenoreceptors can inhibit the splenic response to ischemic brain injury. However, adrenergic inhibitors carry significant side effects, which make them a less advantageous therapeutic alternative.

In some embodiments, treatment can include resection of the spleen, which prevents the egress, trafficking, and infiltration of splenocytes into the brain. In some embodiments, inhibition of the peripheral immune response, through surgical resection of the spleen, can reduce cerebral edema and inflammation and can provide neuronal protection and improved functional outcomes for patients with hypoxic-ischemic and traumatic brain injury. However, the surgical resection of the spleen is not only invasive but also results in the loss of function of the spleen.

In some embodiments, a treatment can be used that temporarily interrupts the brain-spleen inflammatory coupling in the form of preemptive gamma irradiation of the spleen. Irradiation of the spleen can provide the same therapeutic benefits, including reduced inflammation and reduced immune cell infiltration into the area of injury, as that of the resection of the spleen. In some embodiments, the gamma irradiation of the spleen can be generated with a Cobalt-60 (Co-60) irradiation unit, although any suitable gamma radiation generating unit, as are known in the art, can also be employed. FIGS. 1A-B illustrates the splenic irradiation using Co-60 gamma radiation, thereby altering the splenocyte egress to the brain.

Co-60 is a synthetic radioactive isotope of cobalt which is widely used for medical radiotherapy. Co-60 decays by beta emission to a more stable nickel-60 (Ni-60) isotope that emits highly penetrating gamma rays, as illustrated in FIGS. 1A-B. Co-60 irradiation units are clinically more accessible, require less elaborate equipment and staffing, and involve less planning and maintenance when compared to charged particle irradiation devices. In some embodiments, splenic irradiation using an Eldorado Co-60 Teletherapy Unit, which generates gamma rays of 1.17 and 1.33 MeV energy and linear energy transfer of 0.267 KeV/mm, can be used.

Gamma radiation therapy is in clinical use worldwide and is utilized for irradiation of the spleen in other therapeutic techniques. However, alternative modalities are becoming more available. In some embodiments, proton beam therapy that provides highly specific delivery of radiation to small targets can be used. If necessary, proton radiation as an effective alternative to gamma radiation therapy can be employed for irradiation of the spleen, other body components, or whole body irradiation.

In some embodiments, gamma irradiation of the spleen can result in a transient decrease of splenocyte viability, reducing their egress, trafficking, and brain infiltration. The decrease in splenocyte viability can promote the postoperative recovery after surgically-induced brain injury, traumatic brain injury, stroke, various brain disorders, other systemic diseases, and/or other injuries or damage to the body or brain. Additionally, in some embodiments, decreasing the splenocyte viability can ameliorate secondary edema formation, neuroinflammation, and neuronal cell death after surgical brain injury.

Irradiation of the spleen can be preferable over other alternative therapeutic techniques. Current anti-inflammatory pharmacotherapeutics commonly target specific mechanisms of the immune response, such as splenocyte egress, trafficking and brain penetration, or the production of inflammatory molecules. Splenic irradiation, on the other hand, transiently impairs splenocyte viability, therefore targeting the full scope of immune cell-mediated pathophysiology. Therefore, splenic irradiation can be a more effective strategy than available pharmacotherapies. In some embodiments, radiologic splenectomy can be more advantageous and translatable than surgical removal of the spleen because it is noninvasive, well tolerated by patients, and it allows for a regain of splenic functions. Specifically in brain injuries, splenic irradiation can cause reduced brain water content and brain swelling, reduced blood-brain barrier dysfunction, and improved quality of life and outcome.

In some embodiments, splenic irradiation can impair splenocyte viability, egress, and trafficking to the brain, thus ameliorating cerebral edema formation, neuroinflammation, and neuronal cell death after surgically-induced brain injury. Surgically-induced brain injury is the inevitable injury to the normal, yet susceptible, brain that occurs during neurosurgical procedures. In some embodiments, a one-time, low-dose gamma irradiation of the spleen is used as a noninvasive therapy to reduce the detrimental effects of spleen-derived immune cells in the development of surgically-induced brain injury and/or other injuries as disclosed herein and known in the art to be effected by peripheral immune response. Additionally, in some embodiments, the radiation can be applied to other components of the body or several components of the body at the same time. For example, gamma radiation can be utilized to reduce the harmful effects of immune cells activated through a peripheral immune response.

In some embodiments, a radiation treatment can be administered after surgery. The post-surgery treatment can be utilized in conjunction with the pre-treatment of radiation or as the only method of treatment. When utilized with the pre-treatment of radiation, the post-surgery treatment can further reduce the immune response and/or the inflammatory response. The radiation treatment can be administered at any time after surgery for up to about twenty-four (24) hours after surgery to about a week after surgery. In some embodiments, multiple radiation treatments or doses can be utilized post-surgery to reduce the immune response and/or inflammatory response. In some embodiments, only a one time dose is used post-surgery. In some embodiments, the total amount of radiation for treatment used for post-surgery treatment can be 10 Gγ or less.

In some embodiments, the therapies and treatments disclosed herein can be utilized by radiologists, first-responders, primary care physicians, specialists, and other healthcare providers. The therapies and treatments take advantage of irradiation of the spleen for use as a protectant before elective surgeries, including neurosurgery and general surgery, or for use after brain injury occurs, similar to use for treating stroke and traumatic brain injury, as well as other central nervous system diseases that are effected by the implication of peripheral immune cells. In some embodiments, these therapies can speed up the healing process of patients and allow neurosurgeons to take a more aggressive approach in surgery. The reduced inflammation following surgery or injury can aid in the healing process and also reduce risks and costs for health care providers and insurance companies.

In some embodiments, the spleen can be irradiated by a device that can be attached to a patient. The device can be strapped to or attached to the patient with a harness or belt that allows the radiation source to be positioned such that the gamma radiation can target an area or site on the patient such as, for example, the spleen. The device can be removably attached to the harness or belt through an attachment mechanism clip, hook, snap button, bayonet mount, latch, Velcro, or other attachment device. The harness or belt can have a pouch or pocket within or attached to it. The pouch or pocket can be used to house the radiation source within and secure the radiation source to the harness or belt. In other embodiments, the device is permanently attached to and/or formed with the harness or belt. The harness or belt can be attached through bonding with an adhesive or other boding agent, welded together, or otherwise fused together and manufactured to be sold as one single unit.

The device could allow for portability of the irradiation system and enhanced usability in the field. The device can be used in the hospital for primary care of patients with brain injury. Additionally, the device can be used on an ambulance or for first responder care. In other embodiments, a traditional radiation apparatus or machine can be used to produce the gamma radiation at the appropriate specifications for this treatment of the spleen. In some embodiments, any radiation devices including the portable belt or harness device, a portable traditional radiation machine, and/or a stationary radiation machine can be used for gamma irradiation of the spleen for treatment.

Additionally, in some embodiments, if the targeted spleen radiation therapy is unsuccessful in reducing immune cell brain infiltration, whole body radiation can be employed to preemptively blunt the entire population of systemic immune cells.

Preclinical rodent models of traumatic and ischemic brain injury are evaluated for splenocyte infiltration. The splenocytes are shown to penetrate the compromised blood-brain barrier, thus enhancing vasogenic brain edema formation, neuroinflammation, and neuronal cell death. Accordingly, splenectomized animals demonstrate a more pronounced and faster recovery in those models. In some embodiments, brain-spleen inflammatory coupling is evaluated in surgical brain injury similar to the evaluation of traumatic and ischemic brain injury and can suggest that splenocytes play an essential role in the progression of surgically-induced brain injury.

Surgical brain injury induces the release of splenocytes, which infiltrate the brain, thereby causing blood-brain barrier disruption, vasogenic edema formation, and neuroinflammation, ultimately worsening the postoperative sequelae or condition.

The frontal lobe surgical brain injury rat model produces consistent measurable elevations of brain edema and consequent neurofunctional deficits. These models can generate inflammatory molecules such as reactive oxygen species, prostaglandins, and cytokines, which characterize the typical biochemical make-up of surgical brain injury pathophysiology. The model can simulate surgically induced cortical and subcortical brain damage, allowing the controlled exploration of molecular mechanisms and signaling pathways specific for surgical brain injury and differentiated from other underlying brain pathologies.

In some embodiments, gamma irradiation of the spleen, generated via a Co-60 irradiation unit, results in a transient decrease of splenocyte viability, thus reducing their egress, trafficking, and brain infiltration, which promotes postoperative recovery after surgical brain injury. The role of spleen-derived inflammatory cells in the injury progression after surgical brain injury can be established. Effective and preemptive therapeutic strategies for surgically-induced brain injury can support the patient's postoperative recovery.

In some embodiments, consistent with the essential role of spleen derived immune cells in the progression of brain injury, splenectomy can significantly reduce neurodegeneration after cerebral ischemia and traumatic brain injury. In experimental models, rats undergo splenectomy before permanent middle cerebral artery occlusion (MCAO) or other experimental ischemia. The rats that have undergone splenectomy before MCAO demonstrated an approximately 80% decrease in brain infarction volume when compared to non-splenectomized MCAO animals. Furthermore, fewer brain-infiltrated macrophages and granulocytes were found in the splenectomized group. Surgical removal of the spleen, when conducted immediately after traumatic brain injury, reduced cerebral edema and improved cognitive function in rats.

While surgical splenectomy seems to improve the outcome in several brain injury models, it remains an invasive procedure, associated with serious complications such as extensive blood loss, bowel herniation through the incision site, acute mesenteric vein thrombosis, and overwhelming post-splenectomy infections. In contrast, gamma irradiation of the spleen is a noninvasive and effective method to transiently reduce splenocyte viability.

Radiologic splenectomy can reduce spleen size and immune cell count in lymphoepithelial and hemopoietic diseases associated with splenomegaly, e.g., lymphocytic leukemia. It is well tolerated by patients, who predominately report improvement of their symptoms. Spleen-derived immune cells are sensitive to irradiation and undergo apoptosis even at relatively low doses (e.g., 10 Gy or less, e.g., 0.01 Gy to 10 Gy, 0.1 Gy to 10 Gy, 1 Gy to 10 Gy, or the like) of ionizing radiation. Additionally, an irradiation dose of 10 Gy or below does not harm the spleen permanently, but allows for its repopulation with immune cells from primary lymphoid organs. Therefore, gamma irradiation of the spleen is a noninvasive and beneficial therapeutic strategy that is well suited to reduce or prevent surgically-induced brain injury.

Postoperative sequelae of experimental surgical brain injury and consequences of radiologic and surgical splenectomy were evaluated. Adult male Sprague-Dawley rats were used to evaluate the postoperative sequelae of experimental surgical brain injury. Evaluation of the consequences of radiologic and surgical splenectomy in naive or surgical brain injury rats was conducted.

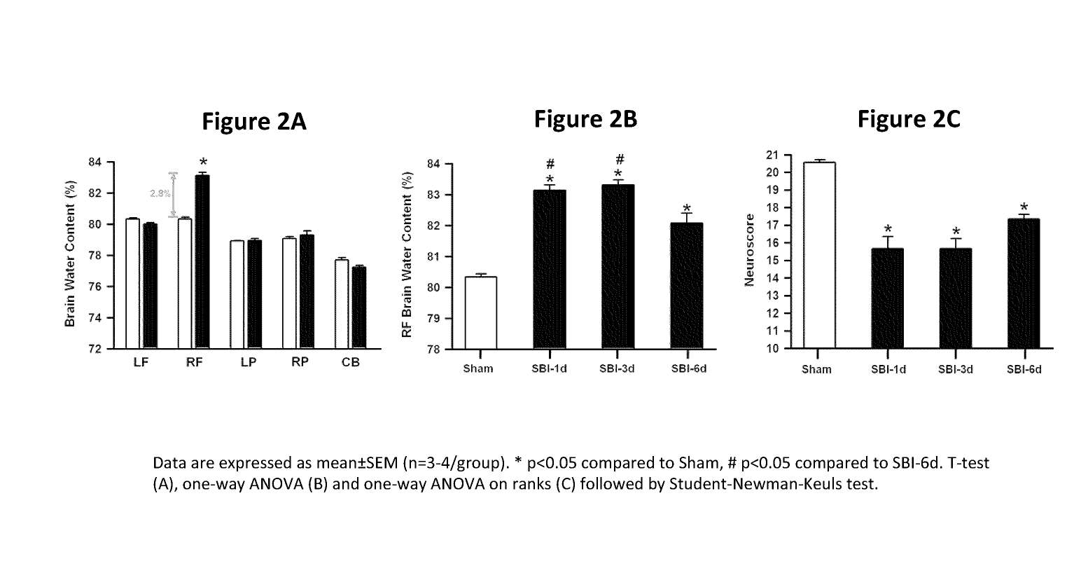

FIGS. 2A-C show the occurrence of brain edema and neurological deficits following surgical brain injury. Brain water content (brain edema) was evaluated using the wet-weight/dry-weight method. Neurological deficits were evaluated with the modified Garcia Neuroscore. FIG. 2A displays the brain water content at one day after surgery. At one day after surgery, surgical brain injury animals showed significantly more brain edema in the right frontal lobe (RF) when compared to sham-operated rats. No differences were seen in the following brain regions: Left frontal (LF), left parietal (LP), right parietal (RP), and cerebellum (CB). The right frontal brain edema remained elevated for six days. The results of the surgical brain injury at one day (SBI-1d), surgical brain injury at three days (SBI-3d), and surgical brain injury at six days (SBI-6d) after surgery are shown in FIG. 2B. FIG. 2C displays the results of the Garcia Neuroscore at one, three, and six days after surgery. Neurological deficits were present on one, three, and six days after surgery. Data are expressed as mean±SEM (n=3-4/group).

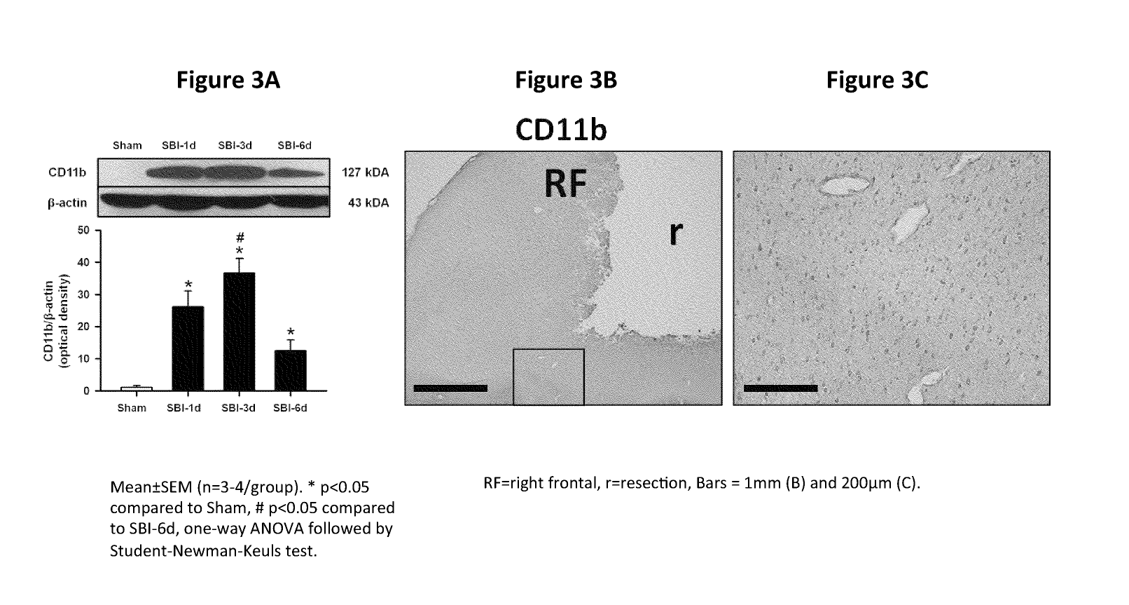

FIGS. 3A-C show CD11b expression and localization following surgical brain injury. Ipsilateral frontal lobes were obtained from sham (post-op day 1) and SBI animals (post-op day 1, 3, and 6) for Western blotting as shown in FIG. 3A. The myeloid lineage-specific cell surface marker CD11b was most highly expressed in the surgical brain injury, SBI, three day group. FIGS. 3B and 3C are representative photomicrographs showing CD11b+ cells (brown) in horizontal brain sections at three days after surgical brain injury-induction. CD11b expression and localization correlates to the presence of leukocytes involved in the innate immune system, including monocytes, granulocytes, macrophages, and natural killer cells.

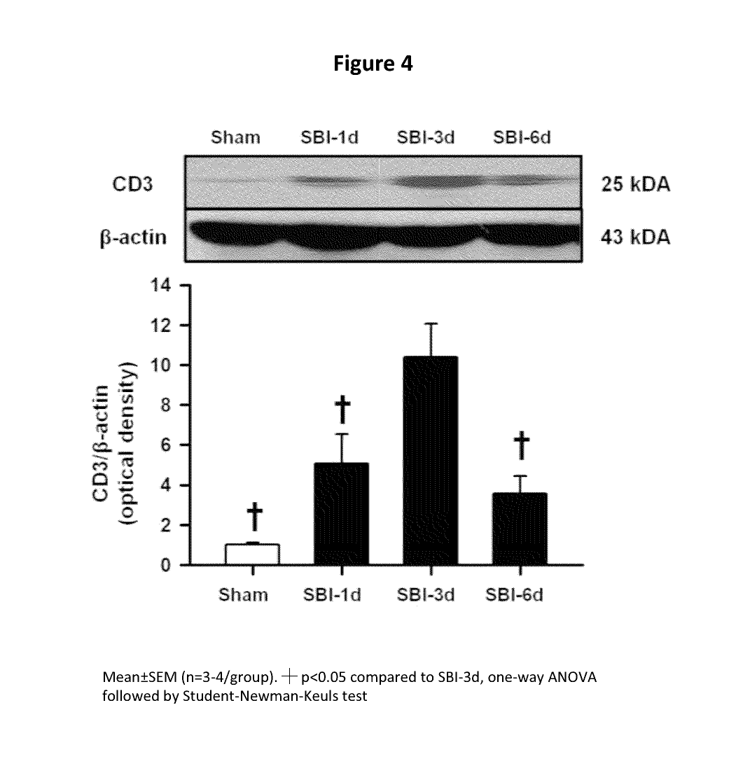

FIG. 4 shows CD3 expression following surgical brain injury. T-lymphocyte specific marker, CD3, expression was significantly higher in the ipsilateral frontal lobe at three days after surgical brain injury (SBI-3d), when compared to all other groups. This shows a significantly higher presence of T-lymphocytes in the ipsilateral frontal lobe at three days after surgical brain injury. Accordingly, the increased CD11b also most highly expressed at three days after surgically-induced brain injury, as well as the increase presence of CD3, correlates to a high immune response at three days after surgical brain injury.

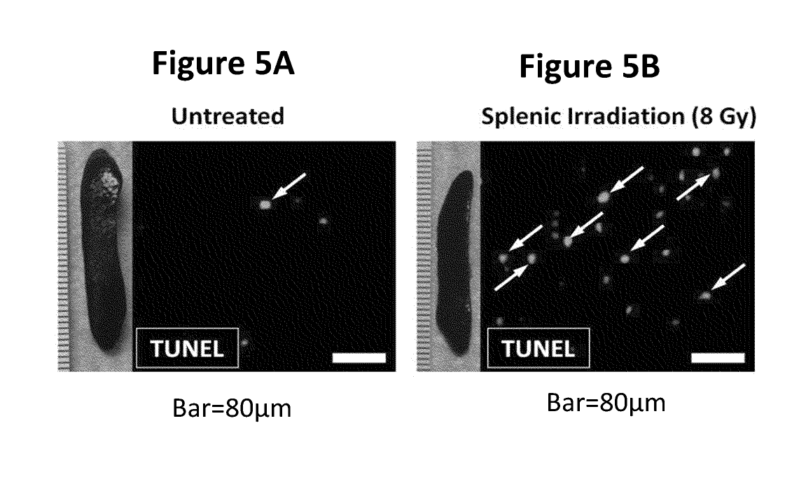

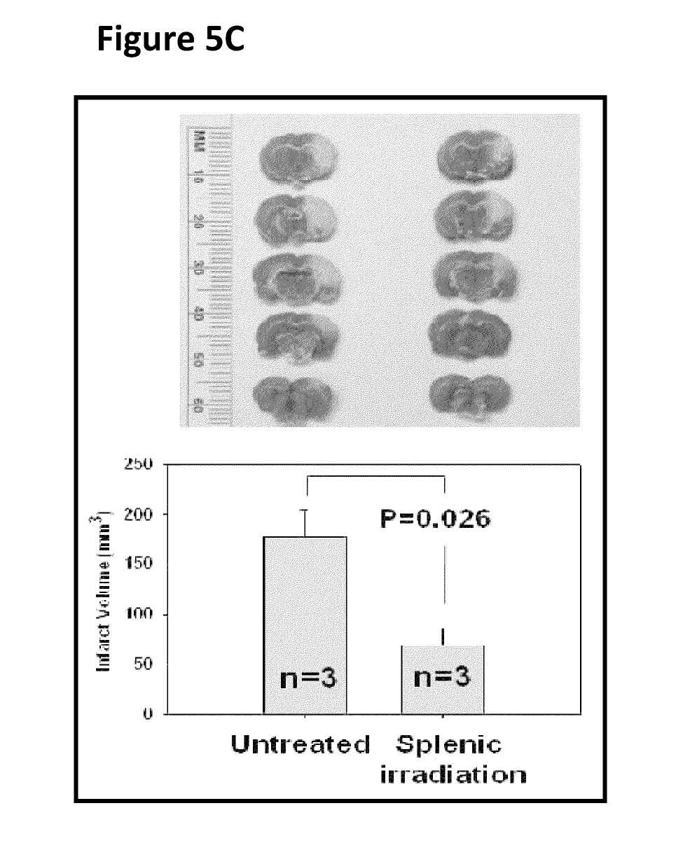

FIGS. 5A-C display gamma irradiation-induced changes in the spleen. Naïve rats were subjected to splenic irradiation. Six days thereafter, spleens were collected, cryosectioned, and examined for presence of cell death via terminal deoxynucleotidyl transferase dUTP nick end labeling (TUNEL). FIG. 5A shows the spleen size and TUNEL-positive cells in untreated animals. FIG. 5B show that the splenic irradiation reduced the spleen size and increased the number of TUNEL-positive cells displayed. The TUNEL-positive cells are noted by the arrows in FIGS. 5A and 5B. This suggests that splenic irradiation reduces the number of viable splenocytes that egress and infiltrate the brain after surgical brain injury. The white bars for FIGS. 5A-B represent a distance of 80 μm. FIG. 5C shows gamma irradiation-induced changes in the spleen with the splenic irradiated samples having a smaller visible size and the volume of the irradiated spleen being reduced.

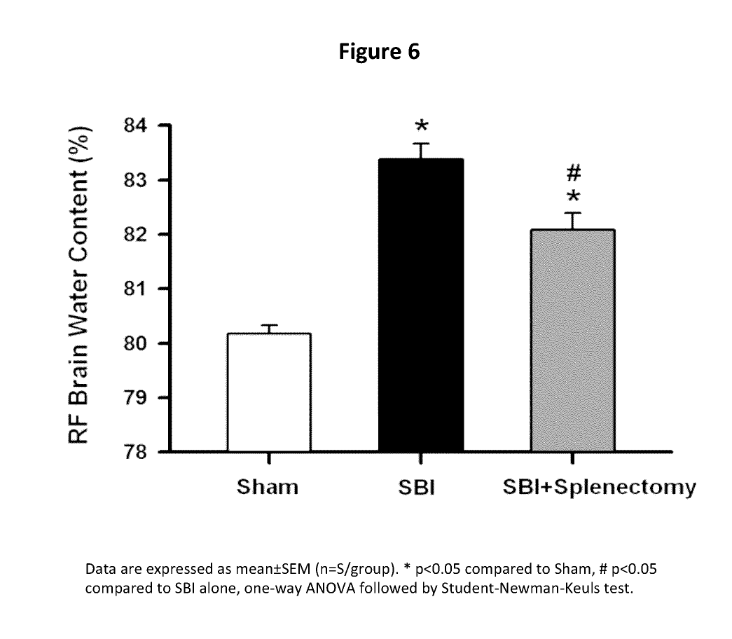

FIG. 6 shows the brain water content in sham surgery, surgical brain injury, and surgical brain injury with splenectomy. Rats were splenectomized one week before surgical brain injury induction. Brain water content was found significantly reduced in the right frontal (RF) lobe of splenectomized rats at three days after surgical brain injury, compared to non-splenectomized rats (SBI group). The reduction in brain water content after surgical brain injury for rats treated with splenectomy shows that the splenectomy reduces brain edema after surgical brain injury at a time, three days, when there is typically a high immune response.

As used herein, reducing the immune response by irradiation of the spleen includes lowering and/or reducing the inflammatory response to a brain injury. This reduction in the inflammatory response will improve patient outcome. Therefore, a successful irradiation treatment includes irradiating the spleen such that the immune cells are damaged so a full inflammatory response is not initiated in the acute phase following brain injury.

### Example 1

### Time-Course of Splenocyte Egress in Surgical Brain Injury Progression

Surgical brain injury stimulates the release of splenocytes, which infiltrate the brain, thereby causing blood-brain barrier disruption, vasogenic edema formation, and neuroinflammation, ultimately worsening the postoperative sequelae. Surgical brain injury causes splenocyte egress, thus decreasing the amount of spleen-resident immune cells and increasing the pool of circulating splenocytes. To determine the amount of spleen-resident and circulating splenocytes after surgical brain injury a rat model is evaluated. Evaluating the time-course of splenocyte egress is essential for the development of specific therapeutic strategies targeting this pathological process.

A total of 48 rats are randomly assigned to surgical brain injury-induction or sham surgery. Sham animals are euthanized at post-op day one, whereas surgical brain injury rats are euthanized at post-op day one, three, or six (n=8/group/time point). Peripheral immune cells in the blood (cardiac puncture) and spleen are quantified by flow cytometry. Whole blood is incubated with erythrocyte lysis buffer and centrifuged to isolate the leukocyte fraction. Spleens are washed in PBS, pressed through a 70 μm cell strainer, centrifuged, incubated with erythrocyte lysis buffer and resuspended. Resultant cell suspensions are counted on a hemocytometer before staining with CD45-PE-Cy7 (pan leukocyte marker), CD11b-PerCP-Cy5.5 (myeloid lineage), CD3-APC (T-lymphocytes), CD19-FITC (B-lymphocytes), CD4-PE (helper T-lymphocytes), CD8-FITC (cytotoxic T-lymphocytes), Ly6G-v450 (neutrophil marker), F4/80-PB (macrophage marker), and NK1.1-FITC (natural killer cells). Flow cytometric analysis is performed using a 7-color MACSquant Analyzer. Data is expressed as mean±SEM. One-way ANOVA followed by the Student-Newman-Keuls test is used for multiple comparisons and the unpaired t-test compares differences between two groups. Statistical significance is defined as p<0.05.

Based on the concentrations of markers in the samples, the resultant levels of peripheral immune cells in the blood and spleen are quantified to determine the time-course and the amount of spleen-resident immune cells and circulating splenocytes.

### Example 2

### Neuropathological Sequelae after Surgical Brain Injury

Surgically-induced brain injury initiates the release of locally-acting cytokines, which causes circulating splenocytes to infiltrate the brain through a compromised blood-brain barrier, thereby enhancing vasogenic brain edema formation, neuroinflammation, and neuronal cell death, ultimately resulting in neurological decline.

A growing body of evidence confirmed the involvement of peripheral immune cells in the progression of acquired brain damage. Accordingly, increased levels of chemotactic cytokines were found in the rat's brain following surgical brain injury-induction. Moreover, preliminary experiments demonstrate increased brain expressions of immune cell markers in surgical brain injury animals. Immune cell infiltration weakens the blood-brain barrier and promotes the formation of brain edema. Brain-infiltrated lymphocytes cause neuronal cell death via contact-dependent mechanisms or by production of TNF-α, which activates extramitochondrial apoptotic pathways via caspase-8.

To evaluate the neuropathological sequelae after surgical brain injury, the types of infiltrating immune cells, their time course of brain infiltration, and their contribution to the development of surgical brain injury are investigated.

A total of 168 rats are randomly assigned to surgical brain injury-induction or sham surgery. Animals are euthanized at post-op day 1, 3, or 6 for the quantification and identification of immune cells in the brain via flow cytometry, measurement of brain edema (n=8/group), Evans blue extravasation assay (n=8/group), Western blotting for immune cell markers and apoptotic cell death (n=8/group), and histology (n=4/group).

The ipsilateral brain hemisphere is minced and passed through a 70 μm cell strainer. The resulting pellets are resuspended in 30% percoll and further centrifuged to obtain an enriched immune cell suspension for flow cytometric analysis. Resultant cell suspensions are stained with CD45-PE-Cy7 (pan leukocyte marker), CD11b-PerCP-Cy5.5 (myeloid lineage), CD3-APC (T-lymphocytes), CD19-FITC (B-lymphocytes), CD4-PE (helper T-lymphocytes), CD8-FITC (cytotoxic T-lymphocytes), Ly6G-v450 (neutrophil marker), F4/80-PB (macrophage marker), and NK1.1-FITC (natural killer cells). Flow cytometric analysis is performed using a 7-color MACSquant Analyzer.

Brains are collected and separated into left and right frontal and parietal lobes as well as the cerebellum for measurement of brain water content. All specimens are weighed on a precise electronic balance and placed in an oven at a temperature of 105° C. for 48 hours. After that, samples are weighed again and brain water content is calculated according to the following formula: [(wet weight−dry weight)/wet weight]×100%. The surgical brain injury model presents with significant increases in brain water content, developing in the ipsilateral frontal lobe within one to three days, as shown by the wet-weight/dry-weight method results. This method is not the gold standard for the measurement of brain water content. When deemed necessary, T2-weighted and diffusion-weighted magnetic resonance imaging to track the progression of brain edema are employed. Furthermore, the wet-weight/dry-weight method does not distinguish between cytotoxic and vasogenic edema. Therefore, Evans blue extravasation assay is included in order to demonstrate progressive changes in the blood-brain barrier integrity after surgical brain injury.

To evaluate the functional integrity of the blood-brain barrier, Evans blue dye (2%; 5 ml/kg) is injected intraperitoneally and allowed to circulate for three hours. Next, rats are terminally anesthetized and transcardially perfused with PBS. Brains are removed and divided into the same regions as for the brain edema study. All specimens are weighed, homogenized in PBS, and centrifuged at 15,000 g for 30 minutes. Next, 0.5 ml of the resultant supernatant is added to an equal volume of trichloroacetic acid (TCA). After overnight incubation and re-centrifugation, supernatants are collected for spectrophotometric quantification of extravasated Evans blue dye at 615 nm as previously described.

Brain specimens (ipsilateral frontal cortex) are collected and processed for Western blot analysis. Equal amounts of extracted protein (50 μg) are separated by SDS-PAGE and transferred onto nitrocellulose membranes, which are blocked and incubated overnight at 4° C. with primary antibodies specific for Iba1 (microglia marker), IL-1β, IL-6, TNF-α (pro-inflammatory cytokines), IL-10 (anti-inflammatory cytokine), and for active forms of caspase-8 and caspase-3 (pro-apoptotic mediators). β-actin is used as loading control. Membranes are washed in TBS and incubated with the appropriate secondary antibodies, before visualization and semi-quantitative analysis. In the event that Western blot analysis of cerebral cytokine expression is unsuccessful, possibly due to a scarce number of target cells, ELISA or RTPCR is employed.

Samples are studied for histology and immunolabeling. Following transcardial perfusion with PBS, brains are collected, formalin fixed, dehydrated, and 10 μm frozen sections are cut on a cryostat (CM3050S; Leica Microsystems). These sections are stained for morphological evaluations (H&E), to determine the extent immune cell infiltration with antibodies as used herein, general cell death (TUNEL), and apoptosis (caspase-3, caspase-8) in neurons (NeuN).

Behavior evaluation is also conducted. All animals are subjected to neurobehavior testing before euthanasia at postop days 1, 3, and 6. A modified 21-point sensorimotor exam is utilized. Sensorimotor testing is graded on a scale of 0 to 3, in a battery of 7 tests: spontaneous activity, side stroking response, vibrissae response, limb symmetry, lateral turning, symmetry of forelimb walking, and climbing. Scores are assigned per test as follows: 0=complete deficit, 1=definite deficit with some function, 2=mild deficit or decreased response, and 3=no evidence of deficit/symmetrical responses. Statistical analysis is performed as described herein, except for behavior data which is analyzed by one-way ANOVA on ranks followed by the Student-Newman-Keuls test. In the event that the behavioral testing yields inconsistent results, cognitive behavioral tests, such as the Morris water maze, are utilized to determine the neurofunctional consequences of our surgical brain injury model.

Surgical brain injury induces acute elevations in brain edema and blood-brain barrier disruption.

### Example 3

### Brain Infiltrating Immune Cells Derived from the Spleen after Surgical Brain Injury

Surgical brain injury induces splenocyte release, trafficking, and subsequent brain infiltration and it can be established that brain infiltrating immune cells derive from the spleen after surgical brain injury. The spleen is targeted, and an evaluation of the proportion of spleen-derived immune cells in the brain after surgical brain injury is conducted.

Five days before surgery, carboxyfluorescein diacetate succinimidyl ester (CFSE) is injected into the rat's spleen. The spleens are externalized under isoflurane anesthesia and 50 μl CFSE (4 mg/ml) is injected into 5 evenly spaced sites along the spleen (250 μl per spleen). Following that, 48 rats, subjected to splenic CFSE injections, are randomly assigned to surgical brain injury-induction or sham surgery. Animals are euthanized at one, three, or six days post-surgical brain injury for quantification of CF SE-positive immune cells in the brain via flow cytometry. Statistical analysis is performed and data is expressed as mean±SEM.

Surgical brain injury results in significant splenic immune cell egress into the blood and trafficking to the brain.

### Example 4

### Role of Spleen-Derived Immune Cells after Surgical Splenectomy on the Development of Surgical Brain Injury

The role of spleen-derived immune cells, by surgical splenectomy, on the development of surgical brain injury is evaluated. A preemptive splenectomy reduces the pool of available immune cells infiltrating the brain after SBI, thereby preventing vasogenic edema formation, neuroinflammation, neuronal cell death, and neurological decline. Surgical splenectomy has been reported to effectively prevent against ischemic and traumatic brain injury. The same therapy is translatable to reduce or prevent surgical brain injury. The splenectomy intervention is an invasive and not translatable procedure but if successful in reducing surgical brain injury will show that inactivating the immune response generated from spleen derived immune cells is a successful treatment for surgical brain injury.

A total of 72 rats are randomly assigned to sham-splenectomy or splenectomy, performed one week prior to surgical brain injury. The experimental groups include surgical brain injury with sham splenectomy and surgical brain injury with splenectomy.

Splenectomy involves isolation and ligation of the splenic blood vessels prior to its removal. Sham splenectomy consists of anesthesia induction, abdominal incision and closure. Animals are euthanized at the most relevant post-op day after surgical brain injury for the quantification and identification of immune cells in the brain via flow cytometry (n=8/group), measurement of brain edema (n=8/group), Evans blue extravasation assay (n=8/group), Western blotting (n=8/group), and histology (n=4/group). Neurofunctional status is evaluated daily following surgical brain injury. Statistical analysis is performed.

Surgical brain injury induces acute elevations in brain edema and blood-brain barrier disruption. Furthermore, surgical brain injury results in significant splenic immune cell egress into the blood and trafficking to the brain. In some embodiments, surgical splenectomy results in significantly less brain infiltration of immune cells and alleviates the neuropathological sequelae after surgical brain injury.

### Example 5

### Evaluation of the Anti-Inflammatory Potential of Splenic Irradiation as a Preoperative Treatment Strategy for Surgical Brain Injury

The anti-inflammatory potential of splenic irradiation as a preoperative treatment strategy for surgical brain injury is evaluated. The targeted gamma rays result in a transient decrease of splenocyte viability, thus reducing overall splenocyte egress and brain infiltration, which promotes postoperative recovery after surgical brain injury.

The impact of gamma irradiation (8 Gy) on spleen morphology and splenocyte viability is evaluated to determine the effectiveness of the irradiation of the spleen. Gamma irradiation of the spleen causes splenocyte cell death, without permanently damaging organ structure and function. The application of gamma rays in a dose of 8 Gy has been reported to effectively reduce splenocyte viability without causing permanent stromal damage or irreversible cessation of splenic functions. Furthermore, it is demonstrated that a dose of 8 Gy gamma irradiation of the spleen is well tolerated, and more effectively reduces spleen size compared to 2 Gy or 5 Gy. The Co-60 unit is used as the gamma ray source. In some embodiments, proton radiation is used as an effective alternative to gamma radiation therapy.

A total of 72 rats are randomly assigned to sham or gamma irradiation (8 Gy) of the spleen. The experimental groups include sham irradiation and splenic irradiation. The CT-assisted splenic irradiation procedure is used on the animals. Rats are sacrificed at 1, 3, 6, and 28 days after gamma irradiation of the spleen or sham irradiation. At these time points, blood and spleens are collected and prepared for splenocyte counts by flow cytometry (n=8/group) and histological evaluation of the spleen (n=4/group). Organ size and weight are determined in non-perfused spleens. Statistical analysis is performed.

Additionally, the effect of splenic irradiation on the development of surgical brain injury is evaluated. The experimental groups include surgical brain injury with sham irradiation and surgical brain injury with splenic irradiation. Preemptive splenic irradiation reduces cerebral infiltration of splenocytes, thereby preventing vasogenic edema formation, neuroinflammation, neuronal cell death, and neurological decline after SBI. The beneficial effects associated with splenic irradiation before surgical brain injury are established.

A total of 72 rats are randomly assigned to sham or gamma irradiation (8 Gy) of the spleen at one day before induction of surgical brain injury. Animals are euthanized at the most relevant day post-surgical brain injury, as determined through techniques and experiments described herein, for the quantification and identification of immune cells in the brain via flow cytometry (n=8/group), measurement of brain edema (n=8/group), Evans blue extravasation (n=8/group), Western blotting (n=8/group), and histology (n=4/group), as described herein. Neurofunctional status is evaluated daily following surgical brain injury. Statistical analysis is performed.

Splenic irradiation results in transient splenic atrophy and cell death of splenocytes, thereby reducing viable immune cells that can egress into the blood and infiltrate the brain after surgical brain injury. Splenic irradiation protects against the surgical brain injury-induced elevations in brain edema, blood-brain barrier disruption, neuroinflammation, and neuronal cell death, which can ameliorate the neurological post-operative decline.

If splenocyte viability is not effectively reduced during surgical brain injury, the effects of spleen irradiation at alternative times before surgical brain injury induction are evaluated and the experimental techniques described herein are repeated under the alternative time conditions. In some embodiments, additional immune cell markers can be utilized to determine changes in pro- and anti-inflammatory phenotypes in these studies, such as Th1/Th2, Treg, and M1/M2 cells.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/of’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or subcombinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

What is claimed is:

1. A method of reducing surgically-induced injury, comprising:
irradiating a target area discrete from a surgical site under gamma radiation from a radiation source, thereby inactivating an immune response generated from the target area; and thereafter
surgically-inducing a primary injury at the surgical site, wherein a secondary injury is reduced as a result of inactivating the immune response from the target area.

2. The method of claim 1, wherein the radiation source is a Cobalt-60 irradiation unit.

3. The method of claim 1, wherein the gamma radiation is of 1.17 MeV or 1.33 MeV energy and exhibits a linear energy transfer of 0.267 KeV/mm.

4. The method of claim 1, wherein the surgical site is a brain.

5. The method of claim 4, wherein the primary injury at the surgical site comprises an acute brain injury.

6. The method of claim 5, wherein the acute brain injury initiates a release of cytokines to stimulate sympathetic adrenergic output.

7. The method of claim 1, wherein the target area is a spleen.

8. The method of claim 7, wherein the inactivated immune response is an inflammatory response by spleen-derived immune cells.

9. The method of claim 1, wherein the secondary injury comprises an edema at the surgical site.

10. The method of claim 1, wherein the target site comprises a plurality of sites in a patient's body.

11. The method of claim 1, wherein reducing the secondary injury further comprises reducing at least one condition selected from the group consisting of edema, neurological defects, blood-brain barrier disruption, neuroinflammation, and neuronal cell death.

12. The method of claim 1, further comprising administering a one-time, low-dose gamma irradiation of the target area, whereby the secondary injury caused by the immune response is further reduced.

13. The method of claim 1, wherein a gamma radiation dose of 10 Gy or less is administered.

14. The method of claim 1, wherein irradiating a target area comprises irradiating a target area discrete from a surgical site under gamma radiation from a radiation source, thereby inactivating an immune response generated from the target area, wherein the target area is a spleen and wherein the surgical site is a brain injury site; and wherein surgically-inducing a primary injury comprises performing a surgical procedure at the brain surgery site, thereby inflicting an acute brain injury at the brain surgery site through performing the surgical procedure, wherein a secondary injury is reduced as a result of inactivating the immune response from the target area, wherein the acute brain injury initiates a release of cytokines to stimulate sympathetic adrenergic output.

15. The method of claim 13, further comprising, after the surgically-inducing the primary injury, administering a single, low-dose gamma irradiation of the target area, whereby the secondary injury caused by the immune response is further reduced.
